(19) United States
(12) Patent Application Publication
(10) Pub. No.: US 2016/0158492 A1
(43) Pub. Date: Jun. 9, 2016

(54) PARTIALLY SHRINKABLE TUBING WITH MULTIPLE LUMENS AND ASSOCIATED METHODS

(71) Applicant: Tephratech LLC, Englewood, CO (US)

(72) Inventors: LeRoy Jutte, Highlands Ranch, CO (US); Leroy D. Geist, Aurora, CO (US)

(21) Appl. No.: 14/964,358

(22) Filed: Dec. 9, 2015

Related U.S. Application Data
(60) Provisional application No. 62/089,783, filed on Dec. 9, 2014.

(51) Int. Cl. A61M 25/00

(52) U.S. Cl. CPC A61M 25/0054; A61M 25/0026; A61M 25/0009; A61M 2025/0024

(57) ABSTRACT

A partially shrinkable tube includes a plurality of lumens. At least one lumen of the plurality of lumens may be shrinkable from an expanded state to an unexpanded state. At least another lumen of the plurality of lumens may remain in an unexpanded state. Methods for manufacturing and using such tubing are also disclosed.

### CROSS-REFERENCE TO RELATED APPLICATION

A claim is hereby made to the benefit of the Dec. 9, 2014, filing date of U.S. Provisional Patent Application 62/089,783, titled METHOD AND APPARATUS FOR A SHRINK TUBING CONSISTING OF MULTIPLE LUMENS THAT WHEN IN THE EXPANDED STATE ALLOW BROADER ACCESS FOR MULTIPLE ITEMS TO BE INSTALLED (“the '783 Provisional Application), pursuant to 35 U.S.C. §119(e). The entire disclosure of the '783 Provisional Application is hereby incorporated herein.

### TECHNICAL FIELD

This disclosure relates generally to shrinkable tubing and, more specifically, to shrinkable tubing with a plurality of lumens. Even more specifically, this disclosure relates to tubing with at least one shrinkable lumen (e.g., a first lumen, etc.), which may be shrunk from an expanded state to an unexpanded state, and another lumen (e.g., a second lumen, etc.) that remains in an unexpanded state. Methods for manufacturing and using such tubing are also disclosed.

### RELATED ART

Tubes that internally carry elongated elements are used for a variety of purposes. Often, such tubes include at least one lumen for carrying an elongated element, and another open lumen that provides a channel through which fluids (e.g., gases, liquids, etc.) may be communicated from one location to another.

Such a multi-lumen tube may be manufactured by extruding the tube around the elongated element. The use of such an extrusion process may, however, be impractical with some types of elongated elements (e.g., the elongated elements may have relatively complex structures, delicate features, be prone to damage when exposed to the temperatures that are used in the extrusion process, etc.). Thus, some elongated elements are assembled with multi-lumen tubes by threading the elongated element into and through an open lumen of the multi-lumen tube. Where the inner diameter of the lumen is not much larger than the outer diameter of the elongated element, assembly in this manner may be extremely difficult. Conversely, where the inner diameter of the lumen is large enough to readily enable introduction of the elongated element therein, space may be wasted and the outer diameter of the multi-lumen tube may be undesirably large.

### SUMMARY

A tube according to this disclosure includes a plurality of lumens. Of the plurality of lumens, one or more dimensions across an interior of at least one lumen (e.g., an inner diameter, etc.) (i.e., a “first lumen”) may be reduced and, therefore, may be referred to as a “shrinkable lumen.” At least another lumen (i.e., a “second lumen”) of the plurality of lumens may retain its dimensions and shape or substantially retain its dimensions and shape (i.e., it may undergo slight dimensional changes (e.g., expansion or contraction due to temperature changes, etc.), it may undergo slight changes in shape (e.g., due to stresses induced thereon by as other parts of the tube, such as a shrinkable lumen, change in dimension and/or shape; due to flattening of the tube under stress, etc.). With at least one shrinkable lumen and at least one fixed lumen, a tube according to this disclosure may be referred to as a “partially shrinkable tube.”

The “shrinkability” of each shrinkable lumen may be imparted by the material of one or more walls of the tube that define at least part of that shrinkable lumen. More specifically, one or more walls that define a shrinkable lumen may be formed from materials that will shrink when subjected to predetermined conditions (e.g., a predetermined temperature, such as a temperature just above a crystalline melting temperature of the material, a specific type of radiation (e.g., ultraviolet (UV) radiation, etc.), etc.).

A shrinkable lumen of a partially shrinkable tube according to this disclosure may be configured to receive an elongated element. Thus, embodiments of tubes that include one or more lumens that carry elongated elements are also disclosed, including embodiments in which the one or more lumens have been shrunk, or have contracted, around the elongated element(s) therein.

In another aspect, this disclosure relates to techniques for manufacturing a partially shrinkable tube that includes a plurality of lumens. Such a method includes extruding the tube from a suitable material (e.g., a cross-linkable polymer, etc.), imparting the material from which the tube is formed with memory (e.g., by cross-linking the cross-linkable polymer, etc.) and expanding at least some portions of the tube (e.g., by heating the material at locations that are to be expanded to a temperature that just exceeds a crystalline melting point of a cross-linked polymer in a vacuum and then rapidly cooling the expanded locations, etc.).

Once a tube has been partially expanded, one or more elongated elements (e.g., elongated medical instruments, etc.) may then be assembled with the partially shrinkable tube. Specifically, one or more elongated elements may be introduced into a shrinkable lumen of the partially shrinkable tube. The partially shrinkable configuration of the tube may enable an end-user to select one or more elongated elements that are to be assembled with the partially shrinkable tube and, in some embodiments, assembly of one or more elongated elements with the partially shrinkable tube may occur just prior to its use, and even at the same site where the assembled tube and elongated element(s) will be used.

After introducing one or more elongated elements into a shrinkable lumen of a partially shrinkable tube, the lumen may be contracted, or shrunk, around each elongated element therein. Such shrinking may be effected by exposing at least a portion of the partially shrinkable tube to conditions that will enable a shrinkable material of the partially shrinkable tube to contract, or shrink (e.g., by applying a sufficient temperature to the shrinkable material, by exposing the shrinkable material to an appropriate wavelength or bandwidth of electromagnetic radiation, etc.). In some embodiments, the shrinkable lumen may return to its original, pre-expanded dimensions, or substantially to those dimensions (e.g., accounting for tolerances in the material to return the shrinkable lumen to its original dimensions, etc.).

With an elongate element in the tube and the tube returned or substantially returned to its original dimensions, the tube may be introduced. In some embodiments, introduction of the tube into the space may occur along a guide wire that extends through a fixed lumen of the tube. With an end of the tube at a desired location within the space, an end of the elongated element may also be present at the desired location, and the fixed lumen may be used to enable further communication between the desired location and an opposite end of the tube.

Other aspects, as well as features and advantages of various aspects, of the disclosed subject matter will become apparent to those of ordinary skill in the art through consideration of the ensuing description, the accompanying drawings and the appended claims.

### BRIEF DESCRIPTION OF THE DRAWINGS

In the drawings:

FIG. 1 is a perspective view of a portion of an embodiment of a partially shrinkable tube according to this disclosure, with the partially shrinkable tube in an expanded, or pre-shrunk, state;

FIG. 2 is a cross-sectional view of the embodiment of partially shrinkable tube shown in FIG. 1;

FIG. 3 is a perspective view of the portion of the embodiment of partially shrinkable tube shown in FIG. 1, with the partially shrinkable tube in an unexpanded state or a contracted, or shrunken, state;

FIG. 4 is a cross-sectional view of the embodiment of partially shrinkable tube shown in FIG. 3;

FIG. 5 is a cross-sectional view of the embodiment of partially shrinkable tube shown in FIG. 1, with one shrinkable lumen in an expanded state and another shrinkable lumen in contracted, or shrunken, state;

FIG. 6 illustrates the introduction of an elongated element into a shrinkable lumen of the embodiment of partially shrinkable tube depicted by FIG. 1;

FIG. 7 depicts an assembly of the elongated element with the embodiment of partially shrinkable tube shown in FIG. 1, with the partially shrinkable tube in its contracted, or shrunken, state; and

FIG. 8 represents an embodiment of introduction of the partially shrinkable tube into an opening, such as the body of a subject, that will accommodate the partially shrinkable tube in its contracted, or shrunken state, but not in its unexpanded state.

### DETAILED DESCRIPTION

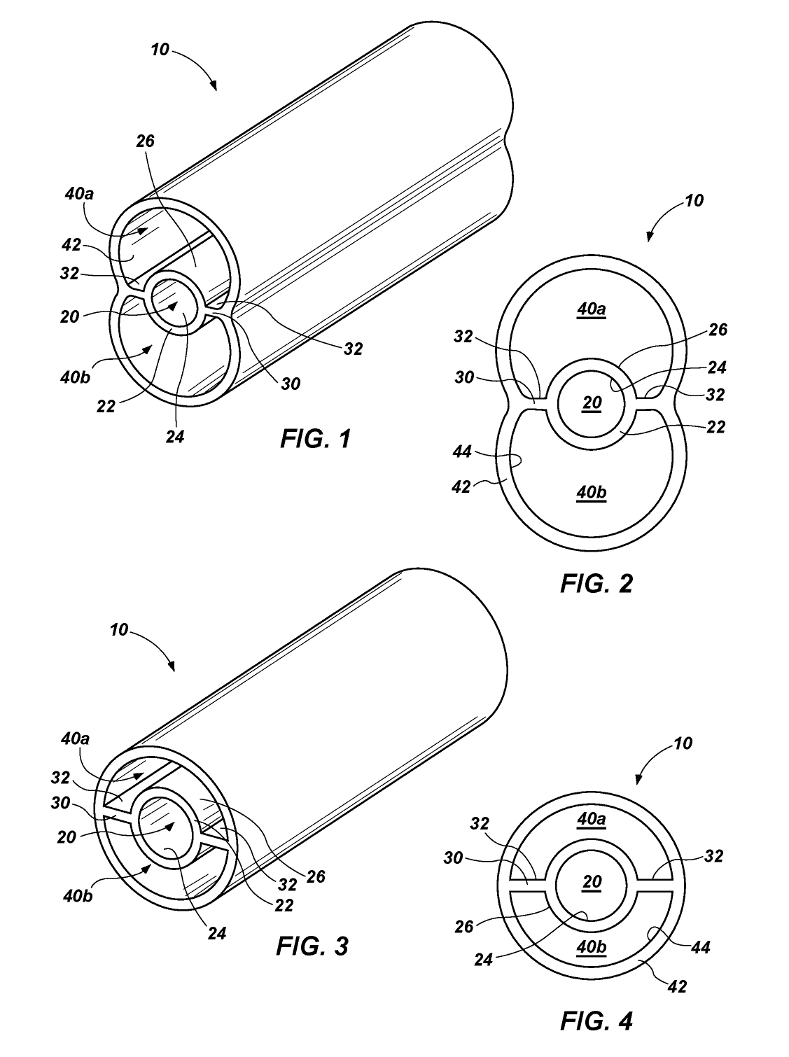

FIGS. 1 and 2 illustrate an embodiment of a partially shrinkable tube 10 according to this disclosure. For the sake of simplicity, the partially shrinkable tube 10 is referred to hereinafter as a “tube.” Although the tube 10 may comprise any of a variety of different types of tubing, in a specific embodiment, the tube 10 may comprise a catheter. The tube 10 includes at least one fixed lumen 20 and at least one shrinkable lumen 40. In the depicted embodiment, the at least one fixed lumen 20 of the tube 10 is a centrally located lumen with a circular cross-section, taken transverse to a length of the fixed lumen 20. The tube 10 includes two shrinkable lumens 40a and 40b on opposite sides of the fixed lumen 20.

More specifically, the fixed lumen 20 of the tube 10 is cylindrical in shape, and is defined by an internal wall 22, which may also be cylindrical in shape. Even more specifically, an inner surface 24 of the internal wall 22 may define the fixed lumen 20 of the tube 10. A pair of connecting ribs 30 extend radially outward from an outer surface 26 of the internal wall 22 to an inner surface 44 of an external wall 42 of the tube 10.

Each shrinkable lumen 40a, 40b of the tube 10 may be defined by a portion of the internal wall 22, a pair of connecting ribs 30 and a portion of the external wall 42. More specifically, each shrinkable lumen 40a, 40b may be defined by a portion of the outer surface 26 of the internal wall 22, opposed surfaces 32 of the pair of connecting ribs 30 and a portion of the inner surface 44 of the external wall 42.

In the illustrated embodiment, in which the tube 10 is in an expanded state, or a pre-shrunk state, the external wall 42 of the tube 10 may be in an expanded state, imparting the shrinkable lumens 40a and 40b with expanded cross-sectional dimensions and, thus, an expanded volume.

As shown in FIGS. 3 and 4, the tube 10 also has an unexpanded state, which may also be referred to as a contracted state, or as a shrunken state, depending upon the tube 10's state of manufacture. When the tube 10 is initially formed (e.g., by extrusion, etc.), before any portion of the tube 10 is expanded, the state of the tube 10 shown in FIGS. 3 and 4 is referred to as an unexpanded state. After expanded portions of the tube 10 (e.g., external wall 42) have been shrunk or otherwise contracted, the state of the tube 10 shown in FIGS. 3 and 4 is referred to as an contracted state or as a shrunken state.

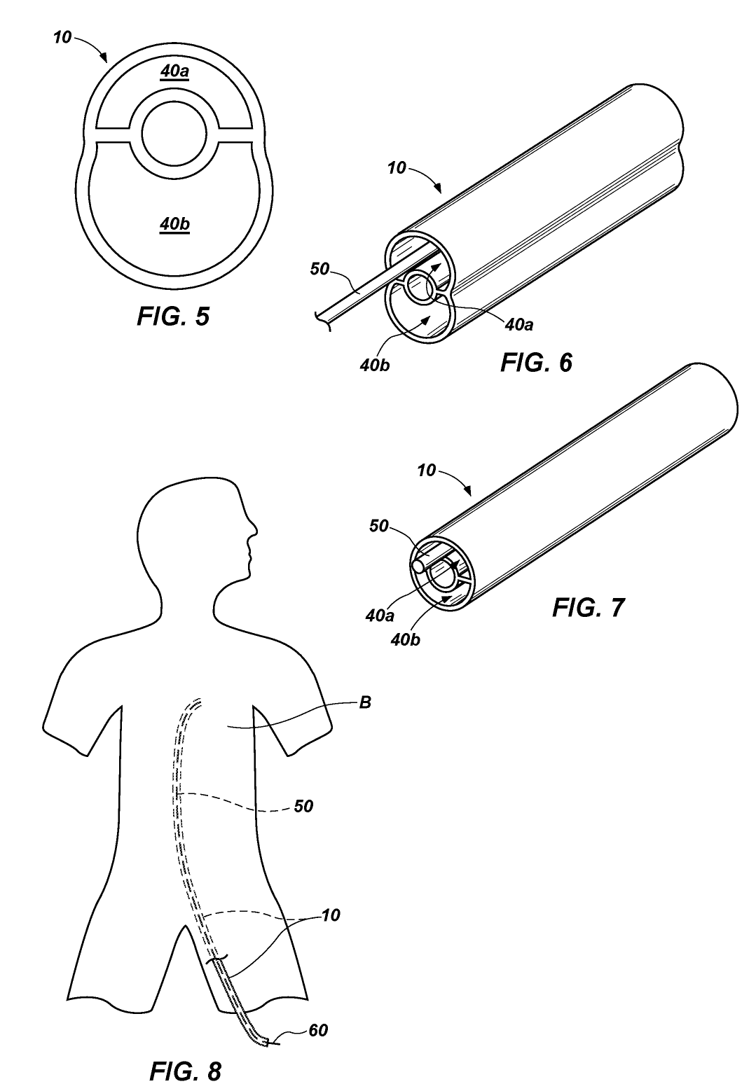

Contraction or shrinking of the tube 10 may comprise shrinking or contraction of the external wall 42 of the tube 10, which may cause the shrinkable lumens 40a and 40b to contract or shrink. In some embodiments where a tube 10 includes a plurality of shrinkable lumens 40a and 40b, one shrinkable lumen 40a may remain unshrunk, while another shrinkable lumen 40b may be shrunk with selectivity, as illustrated by FIG. 5.

In some embodiments, the material from which the external wall 42 and, optionally, each connecting rib 30 is formed may comprise a heat-shrinkable material. Some non-limiting examples of heat-shrinkable materials include, but are not limited to, polyether block amides (e.g., those sold under the PEBAX® trademark by Arkema of Colombes, France, etc.), fluoropolymers (e.g., polytetrafluoroethylene (PTFE), polyvinylidene fluoride (PVDF), fluorinated ethylene propylene (FEP), copolymers of hexafluoropropylene (HFP) and vinylidene fluoride (VDF or VF2), terpolymers of tetrafluoroethylene (TFE), vinylidene fluoride (VDF) and hexafluoropropylene (HFP), perfluoromethylvinylether— (PMVE—) containing specialty polymers, etc.) and polyolefins.

Alternatively, materials that shrink when subjected to other conditions may be used to form the external wall 42 and, optionally, the connecting ribs 30 and/or the internal wall 22. Without limitation, the external wall 42, as well as the connecting ribs 30 and/or the internal wall 22 of a tube 10 according to this disclosure may be manufactured from materials that shrink when exposed to a particular wavelength of electromagnetic radiation (e.g., ultraviolet (UV) radiation, etc.).

The internal wall 22 and, optionally, the connecting ribs 30 may be made from the same material as the external wall 42 or from a different material. When a different material is used to form the internal wall 22 and, optionally, the connecting ribs 30, that material should be compatible with the material of the external wall 42, should be capable of being co-extruded with and securing bonding to the material of the external wall 42 and may have melting point that is higher than a temperature at which the material of the external wall 42 contracts (e.g., a crystalline melting point of the material of the external wall 42, etc.). In some embodiments, the internal wall 22 and, optionally, the connecting ribs 30 may be formed from a different heat-shrinkable material than the heat-shrinkable material that forms the external wall 42.

In some embodiments, the material from which the external wall 42, the connecting ribs 30 and/or the internal wall 22 is formed may include one or more additives. An additive may render the tube 10 more visible under certain circumstances. In some embodiments, the additive may comprise a colorant (e.g., a dye, a phosphorescent material, a fluorescent material, etc.). In other embodiments, the additive may comprise a radio-opaque material (e.g., barium sulfate, bismuth oxide, etc.) or another material that may enable visualization of the tube 10 through other structures (e.g., by fluoroscopy, or via x-ray, etc.).

As an alternative to incorporating an additive into the material of the tube 10, or in addition to incorporating an additive into the material of the tube 10, an additive may be applied to the tube. Additives that may be applied to the tube 10 include colorants, radio-opaque materials, lubricants, antibiotic agents and/or antiviral agents, antiseptic agents, drugs and the like.

A tube according to this disclosure (e.g., tube 10, etc.) may be manufactured by any of a variety of suitable processes and equipment. Without limitation, processes and equipment that are used to extrude multi-lumen tubing (e.g., catheters, etc.). As indicated previously herein, at least the external wall 42 of the tube 10 may be formed from a cross-linkable polymer that may be used in so-called “heat shrink” processes. In embodiments where the external wall 42 and the internal wall 22 of the tube 10 are formed from different materials, the internal wall 22 may be coextruded with the external wall 42.

After a tube 10 has been formed from a suitable cross-linkable material, the material of the tube 10 may be cross-linked. The technique for cross-linking the material of the tube 10 depends at least in part upon the type of material being cross-linked. A few examples of cross-linking techniques include exposing the material to a predetermined type of radiation (e.g., an electron beam, gamma radiation, ultraviolet (UV) light, etc.), exposure of the material to a cross-linking chemical (e.g., a reagent and/or a catalyst, etc.), exposure of the material to moisture, exposure of the material to a specific pH or pH range, and application of heat and/or pressure to a structure made from the material. Cross-linking the material of the tube 10 may impart the tube 10 with memory, which may enable the tube 10 to return to or to return substantially to its original shape and dimensions (i.e., those resulting from the extrusion process) upon shrinkage of expanded portions of the tube 10.

Once the material of the tube 10 is cross-linked, selected portions of the tube 10 may be expanded. In a specific embodiment, the external wall 42 of the tube 10 may be heated to a temperature just above (e.g., about 0.5° above, about 1° C. above, about 2° C. above, about 5° C. above, about 10° C. above, etc.) a crystalline melting point of the material from which the external wall 42 of the tube 10 is formed and subjected to a vacuum to enable the external wall 42 or portions thereof to expand outwardly. Once the external wall 42 has expanded to a desired extent and, optionally, while the tube 10 is still subjected to the vacuum, the external wall 42 may be rapidly cooled (e.g., at a rate of about 0.5° C./s, at a rate of about 1° C./s, at a rate of about 2° C./s, at a rate of about 5° C./s, etc.) to enable it to remain in its expanded state.

In embodiments where the internal wall 22 comprises the same material as the external wall 42, heating of the external wall 42 may be controlled in a manner that enables the external wall 42 to be heated to a temperature suitable for expansion (i.e., the crystalline without heating the internal wall 22 and, optionally, the connecting ribs 30 to that temperature. The duration of time between the point in time when the external wall 42 reaches a sufficient temperature and the point in time when internal wall 22 reaches that same temperature may be relatively small. The extent (i.e., pressure) of the vacuum may be tailored to enable the external wall 42 to expand a desired distance (or the cross-sectional area of the shrinkable lumen 40a, 40b (FIGS. 1 and 2) to expand to a desired extent) in less than that duration of time.

When the internal wall 22 comprises a different material than the external wall 42, the temperature to which the external wall 42 is heated may exceed the crystalline melting point of its material (e.g., a first temperature or a first crystalline melting point), but be less than the crystalline melting point of the material from which the internal wall 22 is formed (e.g., a second temperature or a second crystalline melting point). In embodiments where the difference in these temperatures is sufficient to enable the external wall 42 to be reliably heated and expanded without heating and expanding the internal wall 22, the extent of the vacuum may be carefully controlled to impart each shrinkable lumen 40a, 40b (FIGS. 1 and 2) with a substantially uniform cross-sectional shape and dimensions along its entire length.

The internal wall 22 may be cooled while the external wall 42 is heated. As an example, a wire or another elongated element may be used to prevent undesirably high heating of the internal wall 22 as the external wall 42 is heated. The wire or other elongated instrument may comprise a heat sink, which pulls heat from the internal wall 22. As another alternative, a cooling agent (e.g., air, a gas, a liquid, a solid element, etc.), which may have a temperature that is cool relative to a temperature to which the external wall 42 is heated, may be introduced into the fixed lumen 20 while the external wall 42 is heated.

Once the tube 10 and each of its shrinkable lumens 40a, 40b (FIGS. 1 and 2) have been placed in an expanded state, the cross-sectional area and/or shape, taken along the length of the shrinkable lumen 40a, 40b, may enable one or more elongated elements 50 to be easily and readily inserted, or threaded, into the shrinkable lumen 40a, 40b of the tube 10, as illustrated by FIG. 6. With each elongated element 50 in place within a lumen 40a, 40b of the tube 10, the shrinkable lumen 40a, 40b may be subjected to conditions (e.g., heat, radiation, etc.) that will shrink expanded portions of the external wall 42 and, thus, the shrinkable lumen 40a, 40b around the elongated element 50 therein.

In some embodiments, the cross sectional shape of a lumen 40a, 40b after it has been shrunk may correspond to the cross sectional shape of an elongated element 50 positioned within the lumen. After the lumen 40a, 40b has been shrunk, sufficient space may remain to enable movement of the elongated element 50 along the length of the lumen. Alternatively, the lumen 40a, 40b may shrink onto the elongated element 50, capturing the elongated element 50 within the lumen 40a, 40b, which may prevent longitudinal movement of the elongated element 50 relative to the tube 10.

Prior to contraction of or shrinking the tube 10, a dimension taken transverse to its length (e.g., its outer diameter, etc.) may be too large to enable the tube 10 to be introduced into a desired space. After the shrinkable lumen 40a, 40b and, thus, the tube 10 has been contracted or shrunk, however, that dimension may be small enough to enable insertion of the tube 10 into the desired space.

The tube 10, once an elongated element 50 has been placed in at least one of its shrinkable lumens 40a, 40b (FIGS. 1 and 2) and it has been shrunk or otherwise contracted, may facilitate placement and/or positioning of the elongated element 50, which might otherwise (i.e., without the tube 10) be more difficult, as depicted by FIG. 7. FIG. 8 illustrates a more specific embodiment, in which the tube 10 comprises a catheter and the space into which the tube 10 is introduced is a space within the body B of a subject (e.g., an individual, an animal, etc.). The tube 10 and, thus, the elongated element 50 may be introduced into the body B of a subject along a path defined by a guide wire 60 that was pre-positioned within the body B. More specifically, the guide wire 60 may be introduced into the fixed lumen 20 (FIGS. 3 and 4) of the tube 10 and the tube 10 may then be introduced into the body B of the subject along the guide wire 60, and directed by the guide wire 60 to a desired location within the body B. In embodiments where the tube 10 includes a radio-opaque material, its positioning may be confirmed by fluoroscopy.

A variety different types of elongated elements 50 may be used with a tube according to this disclosure. A few non-limiting examples of suitable elongated elements 50 include, but are not limited to, electrically conductive wires and/or devices that include the same (e.g., sensors, probes, etc.), thermally conductive wires, optical elements (e.g., fiber optics, etc.) and inflatable elements (e.g., balloons, etc.). In some embodiments, an elongated element 50 may be operatively coupled to an apparatus that corresponds to the elongated element 50, such as a monitor (for use with an electrically conductive wire), a heat source (for use with a thermally conductive wire), a camera (for use with fiber optics), a pump (for use with a balloon) or the like.

Although the foregoing description contains many specifics, these should not be construed as limiting the scopes of the inventions recited by any of the appended claims, but merely as providing information pertinent to some specific embodiments that may fall within the scopes of the appended claims. Features from different embodiments may be employed in combination. In addition, other embodiments may also lie within the scopes of the appended claims. All additions to, deletions from and modifications of the disclosed subject matter that fall within the scopes of the claims are to be embraced by the claims.

1. A method for manufacturing heat shrink tubing, comprising:
extruding a tube comprising a plurality of lumens;
selectively expanding at least one lumen of the plurality of lumens of the tube from an original state to an expanded state, at least another lumen of the plurality of lumens remaining in an original state, the original state of the at least another lumen being an original state of the at least another lumen.

2. The method of claim 1, wherein extruding the tube comprises extruding the tube from a cross linkable material.

3. The method of claim 2, further comprising:
cross linking the cross linkable material to provide a cross linked material.

4. The method of claim 3, wherein selectively expanding the at least one lumen of the plurality of lumens comprises:
heating at least a wall defining the at least one lumen to a temperature just above a crystalline melting point of the cross linked material; and
rapidly cooling the wall defining the at least one lumen.

5. The method of claim 4, wherein extruding consists of extruding the tube from the cross linkable material.

6. The method of claim 5, wherein selectively expanding comprises heating exterior surfaces of the tube defining portions of the at least one lumen to a temperature just above the crystalline melting point of the cross linked material without heating interior portions of the tube defining the at least another lumen to a temperature just above the crystalline melting point of the cross linked material.

7. The method of claim 4, wherein extruding comprises:
extruding at least one wall of the at least one lumen from a first material comprising a cross linkable material that, when cross linked, has a first crystalline melting point; and
extruding portions of the tube defining the at least another lumen from a second material having a second melting point, the second melting point of the second material exceeding the first crystalline melting point of the first material.

8. The method of claim 7, wherein extruding portions of the tube defining the at least another lumen from the second material comprises extruding portions of the tube defining the at least another lumen from a second cross linkable material, the second melting point of the second material comprising a second crystalline melting point of a cross linked form of the second material.

9. The method of claim 8, wherein selectively expanding comprises heating the at least one wall to a temperature just above the first crystalline melting point but beneath the second melting point.

10. Heat shrink tubing, comprising:
a first lumen in an unexpanded state; and
a second lumen in an expanded state, the second lumen being shrinkable, upon application of heat to at least one wall defining at least a portion of the second lumen, to an unexpanded state.

11. The heat shrink tubing of claim 10, wherein walls defining the first lumen are located within an interior of the tubing.

12. The heat shrink tubing of claim 11, wherein the at least one wall defining at least a portion of the second lumen is located at an exterior of the tubing.

13. The heat shrink tubing of claim 10, wherein walls defining the first lumen are formed from a different material than the at least one wall defining at least a portion of the second lumen.

14. The heat shrink tubing of claim 10, comprising a catheter having an expanded outer diameter that exceeds a dimension across a space within which the catheter is to be inserted and an unexpanded outer diameter that is less than a dimension across a space within which the catheter is to be inserted.

15. A method for assembling an elongated element with tubing that includes a plurality of lumens, the method comprising:
inserting an elongated element into a first lumen of tubing that includes a plurality of lumens, the first lumen being in an expanded state while inserting the elongated element, the plurality of lumens also including at least one unexpanded lumen; and
shrinking the first lumen from the expanded state to an unexpanded state while the elongated element is located within the first lumen.

16. The method of claim 15, wherein inserting the elongated instrument into the first lumen comprises inserting an elongated medical instrument into the first lumen.

17. The method of claim 16, wherein inserting the elongated medical instrument into the first lumen includes inserting an electronic medical instrument associated with an electronic device into the first lumen.

18. The method of claim 15, wherein shrinking the first lumen from the expanded state to the unexpanded state comprises applying heat to at least one wall defining at least a portion of the first lumen.

19. The method of claim 15, wherein shrinking the first lumen from the expanded state to the unexpanded state comprises capturing the elongated element within the first lumen.

20. The method of claim 15, further comprising:
after shrinking the first lumen from the expanded state to the unexpanded state, introducing an end of the tubing into a space having a dimension that is less than an expanded dimension across the tube when the first lumen is in the expanded state and larger than an unexpanded dimension across the tube when the first lumen is in the unexpanded state.

21. The method of claim 20, wherein introducing comprises inserting an end of a guide wire that protrudes from beyond the space into the second lumen of the tubing and introducing the tubing into the space along a portion of the guide wire that extends into the space.

22. The method of claim 21, wherein introducing comprises introducing a catheter into a body of a subject.
